(19) United States
(12) Patent Application Publication
(10) Pub. No.: US 2025/0033864 A1
(43) Pub. Date: Jan. 30, 2025

(54) PERSONAL HUMIDOR

(71) Applicant: Remon Mansour, El Cajon, CA (US)

(72) Inventor: Remon Mansour, El Cajon, CA (US)

(21) Appl. No.: 18/786,273

(22) Filed: Jul. 26, 2024

Related U.S. Application Data
(60) Provisional application No. 63/516,014, filed on Jul. 27, 2023.

(51) Int. Cl. B65D 85/12; A24F 15/02; A24F 25/02; B65D 1/22; B65D 1/34; B65D 25/04; B65D 25/30; B65D 43/02; B65D 77/04; B65D 81/22

(52) U.S. Cl. CPC B65D 85/12; A24F 15/02; A24F 25/02; B65D 1/22; B65D 1/34; B65D 25/04; B65D 25/30; B65D 43/0202; B65D 77/046; B65D 81/22; B65D 2543/00194

(57) ABSTRACT

A humidor system comprises a container formed of expanded polypropylene (EPP) comprising four side walls and a base, a lid formed of EPP and configured to form an airtight seal with the container; and an accessory tray configured to fit within the container, further including a humidity cutout configured in the base of the container and configured to hold a humidity pack.

### CROSS REFERENCE TO RELATED PATENT APPLICATIONS

This application claims the priority and benefit under 35 U.S.C. § 119 (e) of U.S. Provisional Patent Application Ser. No. 63/516,014, filed Jul. 27, 2023, entitled “PERSONAL HUMIDOR.” U.S. Provisional Patent Application Ser. No. 63/516,014 is herein incorporated by reference in its entirety.

### TECHNICAL FIELD

Embodiments are generally related to tobacco products. Embodiments are further related to humidors. Embodiments are also related to personal humidors. Embodiments are further related to shipping tobacco products. Embodiments are also related to temperature and humidity controlled shipping containers for cigars. Embodiments are additionally related to a systems and methods for securely storing cigars in humidity and temperature controlled humidors.

### BACKGROUND

A humidor is a container configured to store tobacco products including cigars, cigarettes, or pipe tobacco. A humidor is configured to control and maintain the humidity in the container to prevent negative effects on the products inside, which might result from too much or too little humidity. Humidors are configured with systems that store and/or release humidity. In most cases the preferred humidity level for a humidor is between 68%-75%.

Those in the business of cigar sales generally store and or ship cigars in wooden boxes. However, there are currently relatively few options for storing cigars in humidity controlled environments, for personal use and/or for shipping.

For example, cigars are commonly shipped in wooden boxes. Wooden boxes are generally too light to provide rigid protection of the cigars therein, without being unnecessarily heavy and cumbersome to transport. Likewise, cigars tend to transfer oils and aromas to the wooden box. When different cigars are stored in a single wooden box, the aroma and the flavor of the cigars mix, changing the experience for the end consumer.

Furthermore, personal cigar boxes and/or personal humidors tend to be made of wood or plastic, which do not hold up well under stress, exposure to water, exposure to heat, or other such environmental factors. Likewise, most prior art approaches include glue, plastic pieces, hinges, and other aspects prone to failure, or that degrade the product. For example, common issue with wooden humidors is susceptibility to warping, mold growth, or moisture absorption.

Additionally, most traditional humidors require seasoning the wood (moistening it) in order to start the climate control process for cigars. This process is time consuming and inconvenient.

Finally, when a consumer or retailer purchases a cigar or box of cigars, the box the cigars come in is not sufficient to keep them fresh and robust. As a result, consumers and retailers are required to transfer cigars from the box that they ship in, to a humidor. Usually, this is required immediately since the shipping boxes for humidors are not sufficiently temperature and humidity controlled to maintain the quality of the cigars.

Accordingly, there is a need in the art for improved methods, systems, and apparatuses for robust personal humidors and shipping humidors, as disclosed herein.

### SUMMARY

The following summary is provided to facilitate an understanding of some of the innovative features unique to the embodiments disclosed and is not intended to be a full description. A full appreciation of the various aspects of the embodiments can be gained by taking the entire specification, claims, drawings, and abstract as a whole.

It is, therefore, one aspect of the disclosed embodiments to provide a method and system for storing tobacco products.

It is another aspect of the disclosed embodiments to provide a system and apparatus for storing tobacco products for shipping.

It is another aspect of the disclosed embodiments to provide a humidor configured from expanded polypropylene (EPP).

The aforementioned aspects and other objectives and advantages can now be achieved as described herein. In an industry that has primarily relied on wooden humidors, the EPP construction of the humidors disclosed herein offers superior insulation and temperature stability. It effectively address the issue of maintaining optimal temperature and humidity conditions for cigar preservation, thereby enhancing their quality and flavor over extended periods.

The disclosed embodiments also weigh significantly less than traditional wooden humidors and are more robust and insulated than plastic alternatives. The embodiments provide a novel solution to the portability problem by facilitating the easy transportation of up to 80 cigars without the burden of additional weight, a considerable advantage for cigar enthusiasts who travel, or for applications where cigars are transported for personal reasons or for commercial shipping.

The embodiments further address the concern of potential damage during transportation with the introduction of the disclosed impact-resistant, EPP-constructed humidor. This provides enhanced durability and superior protection for cigars, and is a significant improvement over traditional wooden or plastic alternatives.

The disclosed embodiments offer a moisture-resistant design, effectively mitigating common problems with moisture in wooden boxes, and ensuring cigars remain in optima condition.

The disclosed embodiments offer a versatile and customizable storage solution which serves to address the industry's need for flexible storage options. With an adjustable divider, the humidor can be configured to accommodate a user's personal preferences and the size of their cigar collection.

The EPP construction of the humidor systems maintains a consistent internal temperature, eliminating the problem of temperature fluctuations that could hinder the optimal aging conditions for cigars-an issue often encountered with traditional wooden humidors.

The disclosed systems are odor-free, which is advantageous in addressing the issue of flavor interference, which is common in wooden humidors known to impart distinct aromas. The neutral storage environment allows for the natural flavor development of stored cigars.

The disclosed embodiments are formed of a single piece with bevels and edges formed to be easy to clean. In certain embodiments, no moving parts, hinges, or metal parts are included in the system. Maintenance requires only mild soap and water, thereby simplifying maintenance procedures.

The embodiments further provide a specially designed compartment for a humidity control pack. This feature not only solves the problem of maintaining precise humidity control within the humidor but also guarantees the ideal environment for cigar preservation.

For example, in an embodiment a humidor system comprises a container formed of EPP comprising four side walls and a base and a lid formed of EPP and configured to form an airtight seal with the container. In an embodiment, the container is formed of a single piece of material. In an embodiment, the lid is formed of a single piece of material. In an embodiment, the humidor system further comprises a humidity cutout configured in the base of the container and configured to hold a humidity pack. In an embodiment, the humidor system further comprises at least one handle cutout in the sidewall of the container. In an embodiment, the humidor system further comprises a lid lip formed on the container and a sealing lid lip configured to interface with the lid. In an embodiment, the humidor system further comprises an opening cutout formed in the container. In an embodiment, the humidor system further comprises an interior separator.

In an embodiment, a humidor comprises a container formed of EPP comprising four side walls and a base, with a humidity cutout in the base and a lid formed of EPP and configured to form an airtight seal with the container. In an embodiment, the container is formed of a single piece of material and the lid is formed of a single piece of material. In an embodiment, the humidor comprises at least one handle in the sidewall of the container. In an embodiment, the humidor comprises a lid lip formed on the container and a sealing lid lip configured to interface with the lid. In an embodiment, the humidor comprises an interior separator. In an embodiment, the humidor comprises a humidity pack.

In another embodiment, a humidor system comprises a container formed of EPP comprising four side walls and a base, a lid formed of EPP and configured to form an airtight seal with the container, and an accessory tray configured to fit within the container. In an embodiment, the container is formed of a single piece of material, the lid is formed of a single piece of material, and the accessory tray is formed of a single piece of material. In an embodiment, the humidor system further comprises a humidity cutout configured in the base of the container and configured to hold a humidity pack. In an embodiment, the humidor system further comprises a lid lip formed on the container and a sealing lid lip configured to interface with the lid. In an embodiment, the accessory tray further comprises: a single piece body further comprising a first substantially vertical brace, a second substantially vertical brace, and a platform connected to the first substantially vertical brace and the second substantially vertical brace. In an embodiment, the platform further comprises a tray cutout.

It should be appreciated that some or all of the elements of the disclosed system can be formed of EPP as further detailed herein.

### BRIEF DESCRIPTION OF THE DRAWINGS

The accompanying figures, in which like reference numerals refer to identical or functionally similar elements throughout the separate views and which are incorporated in, and form a part of, the specification, further illustrate the embodiments and, together with the detailed description, serve to explain the embodiments disclosed herein.

FIG. 1 depicts a perspective view of a humidor system in accordance with the disclosed embodiments;

FIG. 2 depicts a perspective view of a container, in accordance with the disclosed embodiments;

FIG. 3 depicts an elevation view of a humidor system, in accordance with the disclosed embodiments;

FIG. 4 depicts a side elevation view of a humidor system, in accordance with the disclosed embodiments;

FIG. 5 depicts an elevation view of an interior separator, in accordance with the disclosed embodiments;

FIG. 6 depicts an elevation view of a container, in accordance with the disclosed embodiments;

FIG. 7 depicts a top view of a container, in accordance with the disclosed embodiments;

FIG. 8 depicts a perspective view of a lid, in accordance with the disclosed embodiments;

FIG. 9 depicts an elevation view of a lid, in accordance with the disclosed embodiments;

FIG. 10 depicts a perspective view of a humidor system with an accessory tray, in accordance with the disclosed embodiments; and

FIG. 11 depicts aspects of an accessory tray associated with a humidor system, in accordance with the disclosed embodiments.

### DETAILED DESCRIPTION

The particular values and configurations discussed in the following non-limiting examples can be varied, and are cited merely to illustrate one or more embodiments and are not intended to limit the scope thereof.

Example embodiments will now be described more fully hereinafter with reference to the accompanying drawings, in which illustrative embodiments are shown. The embodiments disclosed herein can be embodied in many different forms and should not be construed as limited to the embodiments set forth herein; rather, these embodiments are provided so that this disclosure will be thorough and complete, and will fully convey the scope of the embodiments to those skilled in the art. Like numbers refer to like elements throughout.

The terminology used herein is for the purpose of describing particular embodiments only and is not intended to be limiting. As used herein, the singular forms “a”, “an”, and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms, such as “and”, “or”, or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures, or characteristics in a plural sense. In addition, the term “based on” may be understood as not necessarily intended to convey an exclusive set of factors and may, instead, allow for existence of additional factors not necessarily expressly described, again, depending at least in part on context.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

The embodiments disclosed herein are drawn to methods, systems, and apparatuses for a transportable humidor, which can serve as a personal humidor or shipping humidor for storing tobacco products in a temperature and humidity controlled environment, in accordance with the disclosed embodiments.

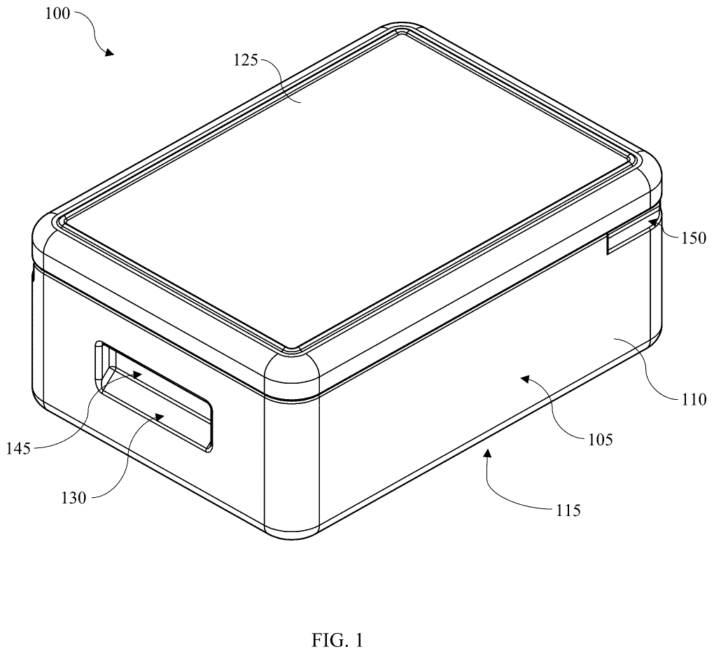

The embodiments disclosed herein are directed to a humidor system such as humidor system 100, illustrated in FIG. 1. The humidor system 100 can generally include a container 105 formed of a four sided expanded polypropylene EPP structure 110 with a base 115, and with an open top 120 and an expanded polypropylene lid 125.

The container 105 can include a cutout 130 on one or more of the sides to serve as a handle 145. The top surface of the container 105 can include an opening cutout 150, that provides a space to separate a lid 125 from the container 105. In certain embodiments, all aspects of the humidor system 100 can be formed of Expanded Polypropylene (EPP).

Expanded polypropylene (EPP) comprises a polypropylene foam material that includes expanded foam particles. Manufacturing with EPP does not require any volatile, organic compounds and the material is recyclable. Furthermore, EPP provides excellent energy absorption, high strength and has a relatively low weight. EPP also provides excellent chemical resistance and thermal insulation. EPP is resistant to oils, chemicals, liquids (including water), and solvents. EPP is nontoxic and tasteless, and is 100% recyclable. Because some or all of the components of the humidor system can be made of EPP, it is ready to use without seasoning or other preparatory steps required for other types of humidors.

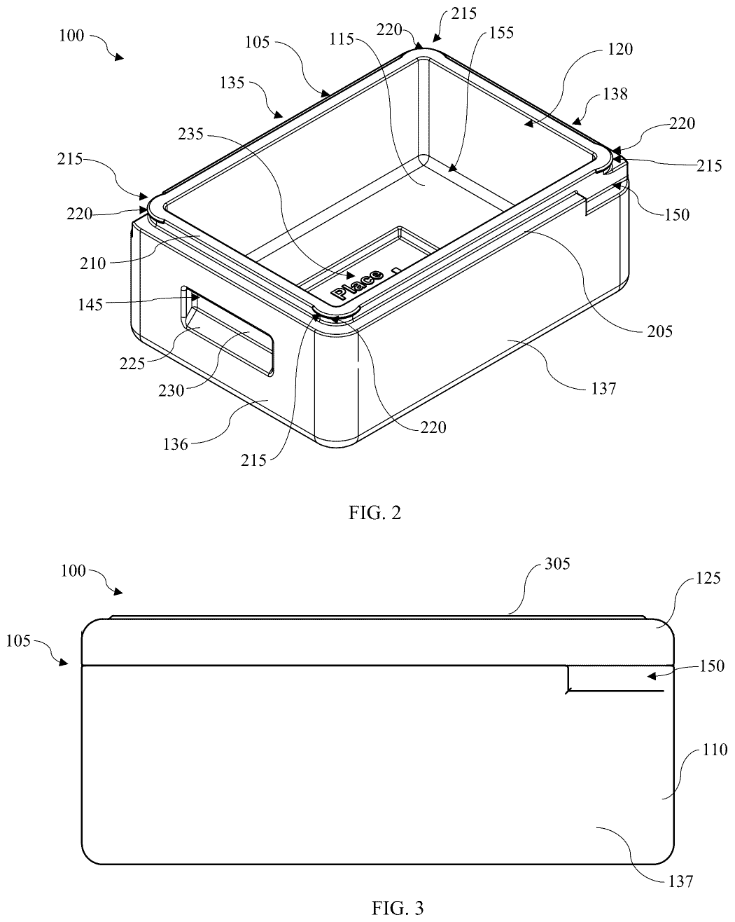

FIG. 2 illustrates aspects of the container 105 associated with the humidor system 100 in accordance with the disclosed embodiments. The container 105 can generally comprise four side walls 135, 136, 137, and 138 and a container base 115. The sidewalls 135, 136, 137, and 138 and base 115 can have an internal bevel 155 at their intersections.

The container 105 can comprise a single piece of EPP, formed to provide the various cutouts and features as further detailed herein. In certain embodiments the EPP can be at least 1 inch thick for all sidewalls 135, 136, 137, and 138 and the base 115 to provide the necessary rigidity and insulation.

The top of the container has a lid lip 205 with a sealing lid lip 210 formed thereon. The lid lip 205 is configured to fit the contour of the lid 125 and the sealing lid lip 210 fits with the lid 125 to create an airtight seal. The corners 215 of the sealing lid lip 210 can have a curved shape configured as corner snaps 220, which engage with the lid 125 to create the solid airtight seal therebetween.

The integrated handles 145 can be formed with an inward and upward sloping face 225 leading to a vertical wall 230 in the side wall, for example side wall 136, which extends upward behind an overhang creating a handle. Other cutout shapes can also be used for handles, without departing from the scope described herein.

The container base 115 can further include a humidity pack cutout 235 which can comprise a rectangular cutout in the container base. In other embodiments other shapes can be used. In some embodiments, the humidity pack cutout 235 can be of a shape and size so as to fit a humidity pack. In some embodiments, the humidity pack can comprise a Boveda® or other such humidity pack configured to maintain the humidity level in the container 105.

In some embodiments, one or more internal separators can be used to separate the container into two or more chambers, in order to store separate flavors or types of tobacco products or cigars, as further detailed herein.

FIG. 3 illustrates an elevation view of the container 105 with the lid 125 attached, in accordance with the disclosed embodiments. As illustrated, the lid 125 can include a stacking lip 305, with can comprise a raised lip formed at or near the edge of the lid 125, and tracking along or near the edge of the top surface of the lid 125. The raised lip 305 is configured to mate with a cutout on the bottom of an additional container 105 such that one or more humidor systems can be stacked.

FIG. 3 further illustrates the opening cutout 150 in the container 105. The opening cutout 150 creates a space between the container sidewall 137 and lid 125 so that the lid 125 can be removed from the container 105.

FIG. 4 illustrates an exemplary side elevation view of the container 105 with the lid 125 attached, in accordance with the disclosed embodiments. It should be appreciated that the opposing side 138 of the container 105 may share some or all of the features illustrated in FIG. 4.

As illustrated in FIG. 4, the lid 125 includes a stacking lip 305, which can comprise a raised lip formed at or near the edge of the lid 125. The raised lip is configured to mate with a cutout on the bottom surface of another container 105 such that the humidor systems 100 can be stacked. FIG. 4 further illustrates the integrated handle 150 formed on the exterior of the sidewall 136 of the container.

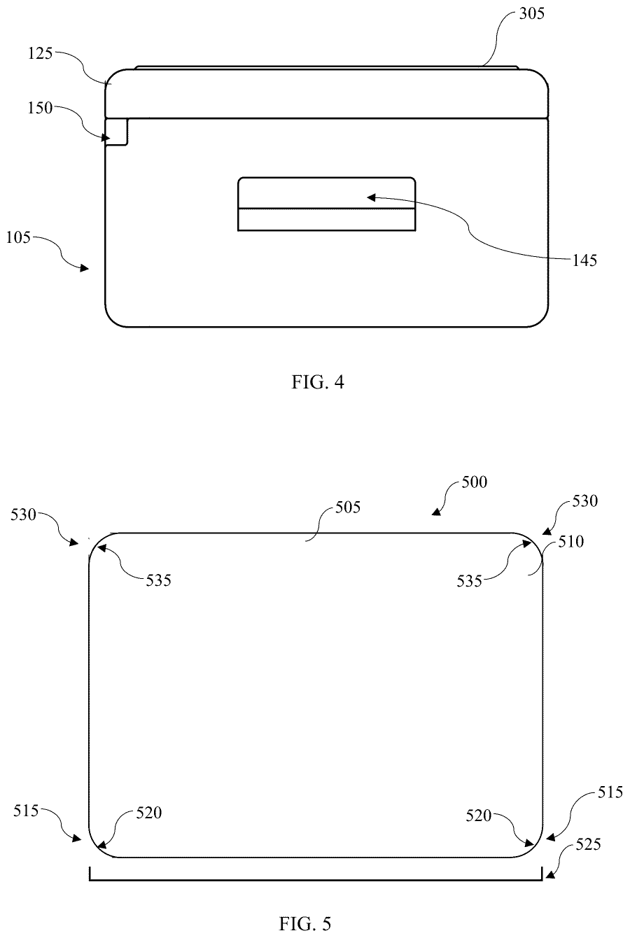

FIG. 5 illustrates an interior separator 500 which can be used to separate the interior volume of the container into separate sections. The aroma and flavor of a cigar can translate among multiple cigars in a single enclosure. For some high quality cigar products, it is desirable not to store them with different cigars, in order to preserve the flavor, aroma, and character of the cigar.

As such, the interior separator 500 can be used to seal one section of the interior volume of the container 105 from another so that the characteristics of the cigars in one section are not imbued on cigars in another section.

The interior separator 500 comprises a separator body 505, with a front surface 510 and rear surface (not shown in FIG. 5). The front surface 510 and rear surface can be substantially flat, or can take other profiles. The length as shown by line 525 of the separator body 505 can be sized to nominally match (or be slightly smaller than) the interior dimension between two opposing walls (e.g. side walls 135 and 137, or side walls 136 and 138).

The bottom corners 515 of the interior separator body 505 can be configured with curves 520. The curves 520 are selected with a radius to fit the curvature of the interior bevel 155 of the container 105. Likewise, the top corners 530 of the interior separator body 505 can be configured with curves 535. The curves 535 are selected with a radius to fit the curvature of the interior bevel of the lid 125 to provide an airtight barrier between the respective sections.

In certain embodiments, the interior separator 500 can be formed of Expanded Polypropylene (EPP).

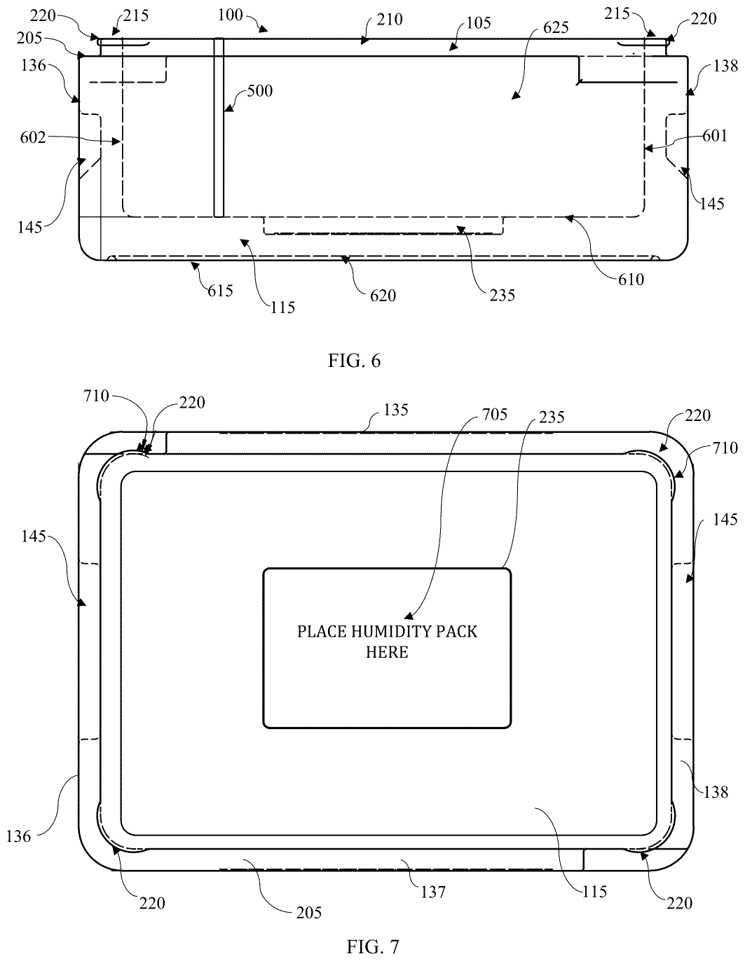

FIG. 6 illustrates an elevation view of a container 105 in accordance with the disclosed embodiments, including details of the humidor system 100. Certain aspects of the container 105 are illustrated as dotted lines for illustrative purposes. This view shows the storage volume 625.

In addition, the container 105 can include sidewalls, including sidewall 136 and sidewall 138, with internal handles 145 formed below a lid lip 205. The sealing lid lip 210 has a smaller perimeter than the lid lip 205, with a curved or rounded profile 220 on the corners 215 to allow the lid 125 to securely connect to the container 105.

The interior sidewall surfaces 601 and 602, and interior base surface 610 are illustrated as a dotted line for reference. The humidity cutout 235 is formed in the base 115 of the container 105.

The exterior surface 615 of the container base 115 can include a stacking cutout 620. The stacking cutout 620 can be sized and positioned to align with the stacking lip 305 on the lid 125 of another humidor system 100. In this way the stacking cutout 620 is configured to mate with the stacking lip 305 on the lid 125, so that multiple humidor systems 100 can be stacked one on top of another.

FIG. 7 illustrates a top plan view of the container 105 associated with the humidor system 100, in accordance with the disclosed embodiments. The container 105 includes a cutout 235 for a humidity pack. In certain embodiments, the cutout can include words 705 like “PLACE HUMIDITY PACK HERE” to indicate that the humidity pack can be placed in the humidity cutout 235.

This view further illustrates the rounded profile 710 of the corner snaps 220. The corner snaps 220 with the rounded or curved shape 710 engage with the lid 125 to create the solid airtight seal between the lid 125 and container 105.

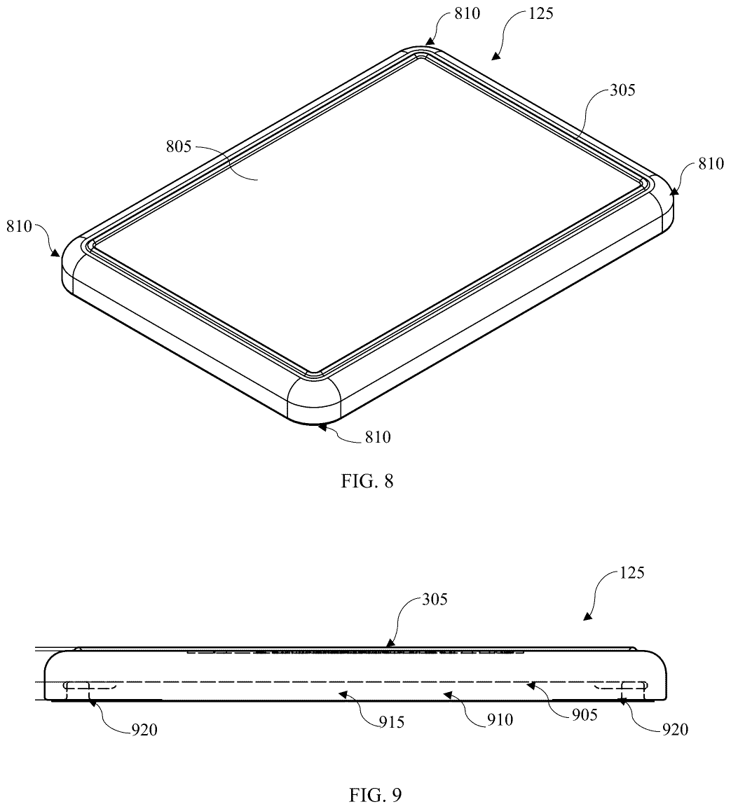

FIG. 8 shows a perspective view of the lid 125 in accordance with the disclosed embodiments. The top surface 805 of the lid 125 can be configured with a stacking lip 305 which can comprise a raised lip formed at or near the edge of the lid 125. The raised lip 305 is configured to mate with a cutout 620 on the bottom of the container 105 such that the containers can be stacked. The corners 810 of the lid 125 can be rounded to match the shape of the container so that the joint between the lid and container is flush and smooth (but for the opening cutout 150).

FIG. 9 illustrates an elevation view of the lid 125. Certain aspects of the lid 125 are illustrated in dotted lines for illustrative purposes. In certain embodiments, the interior surface 905 of the lid 125 can include a raised surface 910 forming an outer lip 915 which is configured to engage with the container 105 and rest on the lid lip 205.

The lid 125 can further include an extruded lid snap 920 configured to engage with the corner snaps 220 on the container 105 to form a solid seal between the lid 125 and container 105.

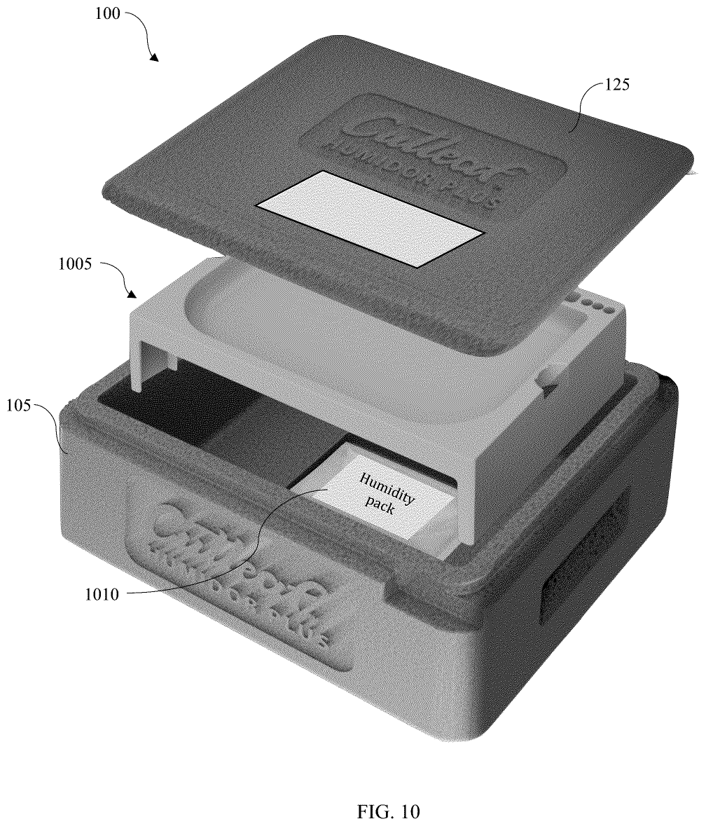

FIG. 10 illustrates aspects of an embodiment of the humidor system 100, incorporating an accessory tray 1005. The accessory tray 1005 can be configured to fit inside the container 105. The accessory tray 1005 is configured to provide space for holding smoking accessories, and or rolling smoking products. The humidity pack 1010 is illustrated in the humidity cutout in the base of the container 105.

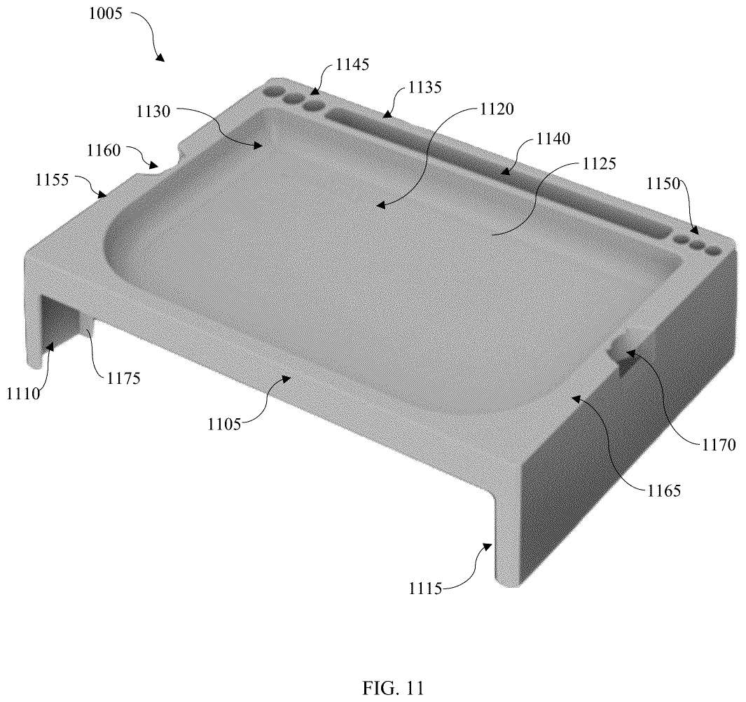

FIG. 11 illustrates aspects of the accessory tray 1005 in accordance with the disclosed embodiments. In certain embodiments the accessory tray 1005 can comprise a single piece body 1105 formed of EPP.

The single piece body 1105 further comprises a first substantially vertical brace 1110 and a second substantially vertical brace 1115 connecting to a platform 1120. The platform can be configured to include a tray cutout 1125. The tray cutout 1125 can include a beveled lower corner 1130 to prevent material from getting stuck in the corner.

The top edge 1135 can further comprise a lateral slot 1140, and a series of larger diameter peg holes 1145 to one side of the lateral slot, and a series of smaller diameter peg holes 1150 to the opposing side of the lateral slot 1140. The lateral slot 1140, larger diameter peg holes 1145 and smaller diameter peg holes 1150 can be used to hold tools, wrappers, grinders, ashtrays, papers, water pipes, and/or smoking products such as cigars or cigarettes.

The first side edge 1155 of the platform 1120 and first vertical brace 1110 can be configured with an excised finger grip slot 1160. Likewise, the second side edge 1165 of the platform 1120 and second vertical brace 1115 can be configured with an excised finger grip slot 1170.

The accessory tray 1005 is configured to fit snugly within the container 105 and to allow the lid 125 to close and form an airtight seal with the container 105, with the accessory tray 1005 therein. As such, the excised finger grip slot 1160 and excised finger grip slot 1170 provide a point of purchase for a user to remove the accessory tray 1005 from the container 105.

The first substantially vertical brace 1110 and a second substantially vertical brace 1115 can each be fitted with a support strut 1175 which lends structural stability to the accessory tray 1005. In embodiments including the accessory tray 1005, the size of the interior separator 500 can be adjusted to ensure the accessory tray 1005 can still fit inside the container 105.

In certain embodiments, all aspects of the accessory tray 1005 can be formed of a single material. In certain embodiments the single piece of material can comprise Expanded Polypropylene (EPP). In other embodiments other materials could be used.

In certain embodiments, all aspects of the humidor system can be made of EPP. The EPP allows the humidor system to provide excellent insulation to the products contained therein, in order to maintain their freshness during transport. In addition, the EPP maintains humidity levels well because the humidor is airtight. The disclosed humidor is configured with insulation in mind. Traditional wood or plastic humidors are not designed or well suited to withstand changes in surrounding temperature. Like wine, changes in temperature negatively impact the performance, taste, quality, etc. of the cigar. The walls of the disclosed humidor can be at least 1 inch thick, and an airtight seal is made between the lid and container. In certain embodiments, the EPP is closed cell, so no air is able to travel through the container walls.

Likewise, the disclosed container and lid can each be formed of a single EPP structure, which is much more robust to damage, provides excellent insulation, is water resistant, is sun proof, is lightweight, is odor proof, offers an airtight seal, prevents oxidation, preserves terpenes, extends shelf life of products stored inside, has no moving parts or pieces, and floats in water making it both dunk proof and safe for products even in the rain.

In certain embodiments, the humidor system can be made completely out of a single material with two parts (container and lid) or three parts (container, lid, and tray). The system requires no assembly, no glues, no screws, and no metal, but still functions as a safe storage container for tobacco products, including an airtight seal.

An aspect of the disclosed embodiments is the dedicated space for the humidity pack. The designated space for the humidity pack allows for the travel of the humidity throughout the humidor box. In prior art approaches that do not include a designated space, humidity may not be evenly distributed.

Another important aspect of the disclosed embodiments is that the container is configured to float. Many cigar enthusiasts spend time in boats. The disclosed humidor is configured to float and to be watertight so that if it falls in water the cigars inside are not damaged. In certain embodiments, the system can be used on fishing trips, for outdoor use, etc.

Another aspect of the disclosed embodiments is that the humidor systems can be used both for cigar shipping and storage. For example, cigars can be shipped in the disclosed humidor systems which are sufficiently insulated and airtight to maintain the optimal temperature and humidity level for the cigars therein. Rushed shipping is not required (as is the case with most current cigar shipping modalities). Additionally, once the cigars reach their retail location or the consumer, the cigars need not be transferred to a different humidor or box, since the disclosed humidor systems are sufficient to maintain the proper temperature and humidity levels to preserve the freshness, aroma, and flavor of the cigars stored therein.

In certain embodiments, the disclosed humidor systems are also configured to be stackable and can be used for shipping purposes. The disclosed humidors are low cost, so that a single type of cigar can be stored in one humidor system, with multiple humidor systems stacked to hold various types/flavors of cigars. For example, it is disadvantageous to mix flavored cigars in the same humidor as non-flavored as the flavors will transfer during aging process.

In other embodiments, the systems can be used for shipping applications. Specifically, the low cost temperature and humidity controlled humidor dramatically improves on standard shipping, which must be expedited because standard shipping freight is not configured to maintain cigars in an ideal environment.

Based on the foregoing, it can be appreciated that a number of embodiments, preferred and alternative, are disclosed herein. For example, in an embodiment a humidor system comprises a container formed of EPP comprising four side walls and a base and a lid formed of EPP and configured to form an airtight seal with the container. In an embodiment, the container is formed of a single piece of material. In an embodiment, the lid is formed of a single piece of material. In an embodiment, the humidor system further comprises a humidity cutout configured in the base of the container and configured to hold a humidity pack. In an embodiment, the humidor system further comprises at least one handle cutout in the sidewall of the container. In an embodiment, the humidor system further comprises a lid lip formed on the container and a sealing lid lip configured to interface with the lid. In an embodiment, the humidor system further comprises an opening cutout formed in the container. In an embodiment, the humidor system further comprises an interior separator.

In an embodiment, a humidor comprises a container formed of EPP comprising four side walls and a base, with a humidity cutout in the base and a lid formed of EPP and configured to form an airtight seal with the container. In an embodiment, the container is formed of a single piece of material and the lid is formed of a single piece of material. In an embodiment, the humidor comprises at least one handle in the sidewall of the container. In an embodiment, the humidor comprises a lid lip formed on the container and a sealing lid lip configured to interface with the lid. In an embodiment, the humidor comprises an interior separator. In an embodiment, the humidor comprises a humidity pack.

In another embodiment, a humidor system comprises a container formed of EPP comprising four side walls and a base, a lid formed of EPP and configured to form an airtight seal with the container, and an accessory tray configured to fit within the container. In an embodiment, the container is formed of a single piece of material, the lid is formed of a single piece of material, and the accessory tray is formed of a single piece of material. In an embodiment, the humidor system further comprises a humidity cutout configured in the base of the container and configured to hold a humidity pack. In an embodiment, the humidor system further comprises a lid lip formed on the container and a sealing lid lip configured to interface with the lid. In an embodiment, the accessory tray further comprises: a single piece body further comprising a first substantially vertical brace, a second substantially vertical brace, and a platform connected to the first substantially vertical brace and the second substantially vertical brace. In an embodiment, the platform further comprises a tray cutout.

It will be appreciated that variations of the above-disclosed and other features and functions, or alternatives thereof, may be desirably combined into many other different systems or applications. Also, various presently unforeseen or unanticipated alternatives, modifications, variations, or improvements therein may be subsequently made by those skilled in the art which are also intended to be encompassed by the following claims.

What is claimed is:

1. A humidor system comprising:
a container formed of EPP comprising four side walls and a base; and
a lid formed of EPP and configured to form an airtight seal with the container.

2. The humidor system of claim 1 wherein the container is formed of a single piece of material.

3. The humidor of claim 1 wherein the lid is formed of a single piece of material.

4. The humidor system of claim 1 further comprising:
a humidity cutout configured in the base of the container and configured to hold a humidity pack.

5. The humidor system of claim 1 further comprising:
at least one handle cutout in the sidewall of the container.

6. The humidor system of claim 1 further comprising:
a lid lip formed on the container; and
a sealing lid lip configured to interface with the lid.

7. The humidor system of claim 1 further comprising:
an opening cutout formed in the container.

8. The humidor system of claim 1 further comprising:
an interior separator.

9. A humidor comprising:
a container formed of EPP comprising four side walls and a base, with a humidity cutout in the base; and
a lid formed of EPP and configured to form an airtight seal with the container.

10. The humidor of claim 9 wherein the container is formed of a single piece of material and the lid is formed of a single piece of material.

11. The humidor of claim 9 further comprising:
at least one handle in the sidewall of the container.

12. The humidor of claim 9 further comprising:
a lid lip formed on the container; and
a sealing lid lip configured to interface with the lid.

13. The humidor of claim 9 further comprising:
an interior separator.

14. The humidor of claim 9 further comprising:
a humidity pack.

15. A humidor system comprising:
a container formed of EPP comprising four side walls and a base;
a lid formed of EPP and configured to form an airtight seal with the container; and
an accessory tray configured to fit within the container.

16. The humidor system of claim 15 wherein the container is formed of a single piece of material, the lid is formed of a single piece of material, and the accessory tray is formed of a single piece of material.

17. The humidor system of claim 15 further comprising:
a humidity cutout configured in the base of the container and configured to hold a humidity pack.

18. The humidor system of claim 15 further comprising:
a lid lip formed on the container; and
a sealing lid lip configured to interface with the lid.

19. The humidor system of claim 15, wherein the accessory tray further comprises:
a single piece body further comprising:
a first substantially vertical brace;
a second substantially vertical brace;
and a platform connected to the first substantially vertical brace and the second substantially vertical brace.

20. The humidor system of claim 19 wherein the platform further comprises:
a tray cutout.
